(19) United States
(12) Patent Application Publication
(10) Pub. No.: US 2011/0006228 A1
(43) Pub. Date: Jan. 13, 2011

(54) Sterra-Sponge

(76) Inventor: Adam Perry Tow, Osprey, FL (US)

Correspondence Address: WILSON DANIEL SWAYZE, JR., 3804 CLEARWATER CT., PLANO, TX, 75025, US

(21) Appl. No.: 12/783,293

(22) Filed: May 19, 2010

Related U.S. Application Data
(60) Provisional application No. 61/216,585, filed on May 19, 2009.

(51) Int. Cl. A61L 2/00

(52) U.S. Cl. 250/504 R; 250/493.1

(57) ABSTRACT

A cleaning and sterilizing device to clean and sterilize a work surface may include a top member and a bottom member to cooperate with the top member to provide a container for a sterilizing light device to provide sterilizing light for the work surface. The top member may include includes a first enclosure and the bottom member includes a second enclosure to cooperate with the first enclosure to substantially cover a light bulb to generate a sterilizing light for the work surface and a cleaning apparatus may be detachably connected to the bottom member to simultaneously clean the work surface as the sterilizing light is sterilizing the work surface. The sterilizing light may be an ultraviolet light, and the cleaning apparatus may include a sponge detachably connected to the bottom member. The sponge may be connected to the bottom member with a Velcro hooks and loops, and the sponge may be connected to the bottom surface of the bottom member. The bottom member may include a dividing wall to separate the sponge from the sterilizing light.

### PRIORITY

The present invention claims priority under 35 USC section 119 based upon a provisional application with a Ser. No. of 61/216,585 which was filed on May 19, 2009.

### FIELD OF THE INVENTION

The present invention relates to a device for generating a clean and sterile surface.

### BACKGROUND

The housewife of today generally requires a clean and sterile surface especially in the kitchen and bathroom. Usually, strong chemicals are used to obtain the clean and sterile surface. However, these chemicals may not always be desirable to use in a household. Generally, a residue may be left from these chemicals which may enter into the food supply. Furthermore, these chemicals may partially evaporate into the surrounding air, and consequently, these chemicals may be breathed by the occupants and these chemicals may be harmful.

### SUMMARY

A cleaning and sterilizing device to clean and sterilize a work surface may include a top member and a bottom member to cooperate with the top member to provide a container for a sterilizing light device to provide sterilizing light for the work surface.

The top member may include a first enclosure and the bottom member may include a second enclosure to cooperate with the first enclosure to substantially cover a light bulb to generate a sterilizing light for the work surface, and a cleaning apparatus may be detachably connected to the bottom member to simultaneously clean the work surface as the sterilizing light is sterilizing the work surface.

The sterilizing light may be an ultraviolet light, and the cleaning apparatus may include a sponge detachably connected to the bottom member.

The sponge may be connected to the bottom member with a Velcro hooks and loops, and the sponge may be connected to the bottom surface of the bottom member.

The bottom member may include a dividing wall to separate the sponge from the sterilizing light.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the top member of the present invention;

FIG. 2 illustrates a perspective view of the top member of the present invention;

FIG. 3 illustrates a bottom view of the top member of the present invention;

FIG. 4 illustrates a side view of the top member of the present invention;

FIG. 5 illustrates a perspective view of the top member and the handle of the present invention;

FIG. 6 illustrates a side view of the top member and handle of the present invention

FIG. 7 illustrates a perspective view of the bottom member of the present invention;

FIG. 8 illustrates a perspective view of the bottom member of the present invention;

FIG. 9 illustrates a bottom view of the bottom member of the present invention;

FIG. 10 illustrates a side view of the bottom member of the present invention;

FIG. 11 illustrates a perspective view of the bottom member of the present invention;

FIG. 12 illustrates a side view of the bottom member of the present invention;

FIG. 13 illustrates a perspective view of the cleaning and sterilizing device of the present invention;

FIG. 14 illustrates a perspective view of the cleaning and sterilizing device of the present invention;

FIG. 15 illustrates a perspective view of the cleaning and sterilizing device of the present invention;

FIG. 16 illustrates a perspective view of another top member of the present invention;

FIG. 17 illustrates a perspective view of another top member of the present invention;

FIG. 18 illustrates a bottom view of the top member of the present invention;

FIG. 19 illustrates a side view of the top member of the present invention;

FIG. 20 illustrates a perspective view of the top member of the present invention;

FIG. 21 illustrates an end view of the top member of the present invention;

FIG. 22 illustrates a perspective view of the bottom member of the present invention;

FIG. 23 illustrates a perspective view of the bottom member of the present invention;

FIG. 24 illustrates a bottom view of the bottom member of the present invention;

FIG. 25 illustrates a side view of the bottom member of the present invention;

FIG. 26 illustrates a perspective view of the bottom member of the present invention;

FIG. 27 illustrates a side view of the bottom member of the present invention;

FIG. 28 illustrates a perspective view of the top member, the bottom member and the cleaning apparatus of the present invention;

FIG. 29 illustrates a perspective view of the top member, the bottom member and the cleaning apparatus of the present invention;

FIG. 30 illustrates a side view of the top member, the bottom member and the cleaning apparatus of the present invention;

FIG. 31 illustrates a perspective view of another top member of the present invention;

FIG. 32 illustrates a perspective view of the top member of the present invention;

FIG. 33 illustrates a bottom view of the top member of the present invention;

FIG. 34 illustrates a side view of the top member of the present invention;

FIG. 35 illustrates a perspective view of the top member of the present invention;

FIG. 36 illustrates a side view of the top member of the present invention;

FIG. 37 illustrates a perspective view of another bottom member of the present invention;

FIG. 38 illustrates a bottom view of the bottom member of the present invention;

FIG. 39 illustrates a circuit diagram of the sterilizing light device of the present invention;

FIG. 40 illustrates a perspective view of the top member of the present invention;

FIG. 41 illustrates a perspective view of the bottom member of the present invention;

FIG. 42 illustrates a perspective view of the bottom member of the present invention;

FIG. 43 illustrates a perspective view of the top member and the bottom member of the present invention.

### DETAILED DESCRIPTION

The present invention allows the user to connect a cleaning apparatus such as a sponge to be detachably connected for example with Velcro to the bottom surface of the bottom wall 161 of the cleaning apparatus and allows the user to activate a sterilizing light device 109 to generate ultraviolet light which sterilizes a work surface 201 while the work surface 201 is being cleaned with a sponge. A dividing wall 163 divides the sponge from the source of ultraviolet light.

The present invention provides a device which may simultaneously clean and sterilize a surface without the need for strong chemicals. The present invention utilizes a sterilizing light in order to provide a sterilize surface and may include a cleaning apparatus such as a sponge in order to clean the surface from the debris which may have accumulated on the surface. The present invention kills the germs and subsequently wipes them away to eliminate substantially any trace of the germs. For example, and an endo-toxic bacteria such as salmonella may be killed by ultraviolet light, but the bacteria remains harmful to humans even when dead because the outer layer (LPS lipopolysaccaride) may be unaffected by the UV light which may be 253.7 nm wavelength or other wavelengths, and is toxic to humans. The present invention kills such bacteria, and substantially eliminates the remains of the bacteria to remove the toxin. The cleaning apparatus may use simple cleaning chemicals such as plain soap which generally has little adverse effect on the users of the cleaning apparatus.

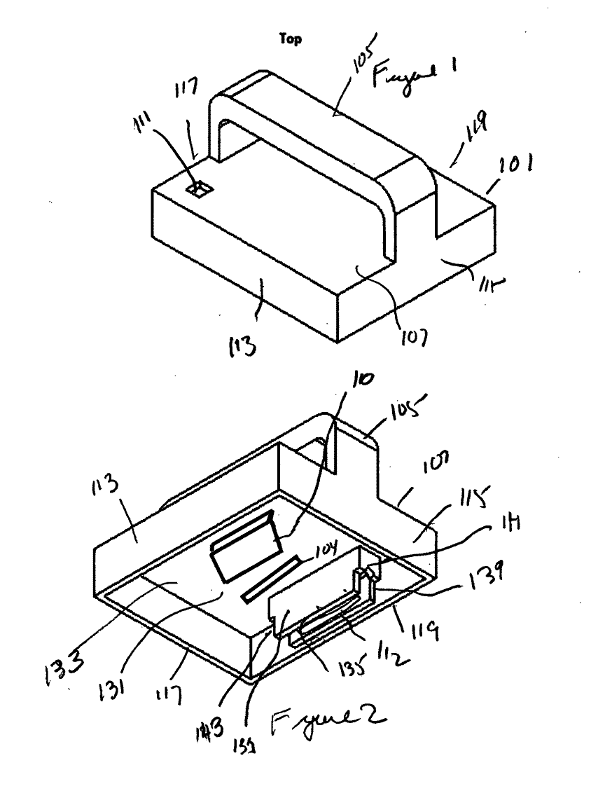

FIG. 1 illustrates a top perspective view of the top member 101 of the cleaning and sterilizing device 100 which may include a top wall 103 and a handle 105 which may be U-shaped and may be connected to the top surface 107 of the top wall 103 in order to allow the user to carry the cleaning and sterilizing device 100. The top member 101 of the cleaning and sterilizing device 100 may include an aperture 111 which may extend through the top wall 103 to cooperate with a button to activate the sterilizing light device 109. The top wall may be connected to a front wall 113 and may be connected to a first sidewall 115 and may be connected to a second sidewall 117 and may be connected to a rear wall 119.

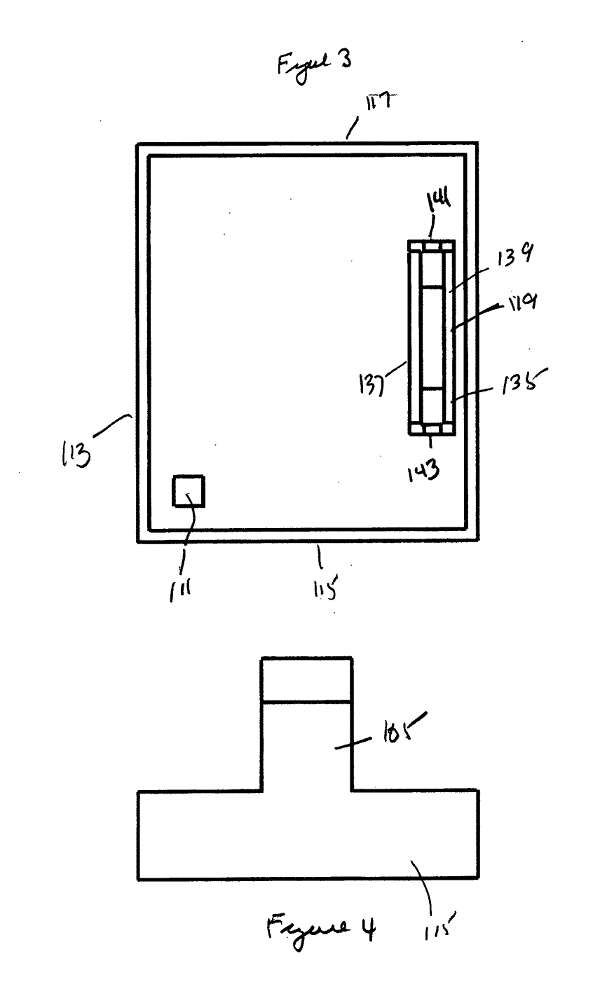

FIG. 2 illustrates that the front wall 113 may be connected to the first sidewall 115 which may be connected to the front wall 113 and the rear wall 119 and which may be opposed to the second side wall 117 which may be connected to the front wall 113 and the rear wall 119. The front wall 113, the rear wall 119, the first sidewall 115 and the second sidewall 119 may define an aperture 113 to cooperate with the bottom member 151.

FIG. 2 additionally illustrates a first enclosure 135 to cooperate with a light bulb to generate ultraviolet light of the sterile light device 109 which may include a circuit 104 which may be connected to a light bulb 112 to admit ultraviolet light and which may be connected to a battery 110 for power and which may be positioned between a first sidewall 137 and an opposed second sidewall 139 which may be connected to a first end wall 141 and an opposed second end wall 143. The first sidewall 137 and the opposed second sidewall 139 may extend downward beyond the first end wall 141 and the second end wall 143. The first sidewall 137, the second sidewall 139, the first end wall 141 and the second end wall 143 defined an enclosure for the sterilizing light of the sterilizing light device 109.

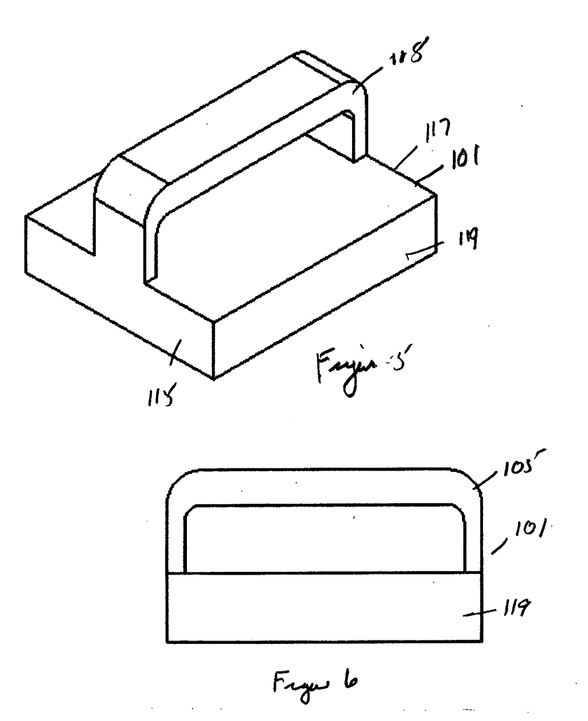

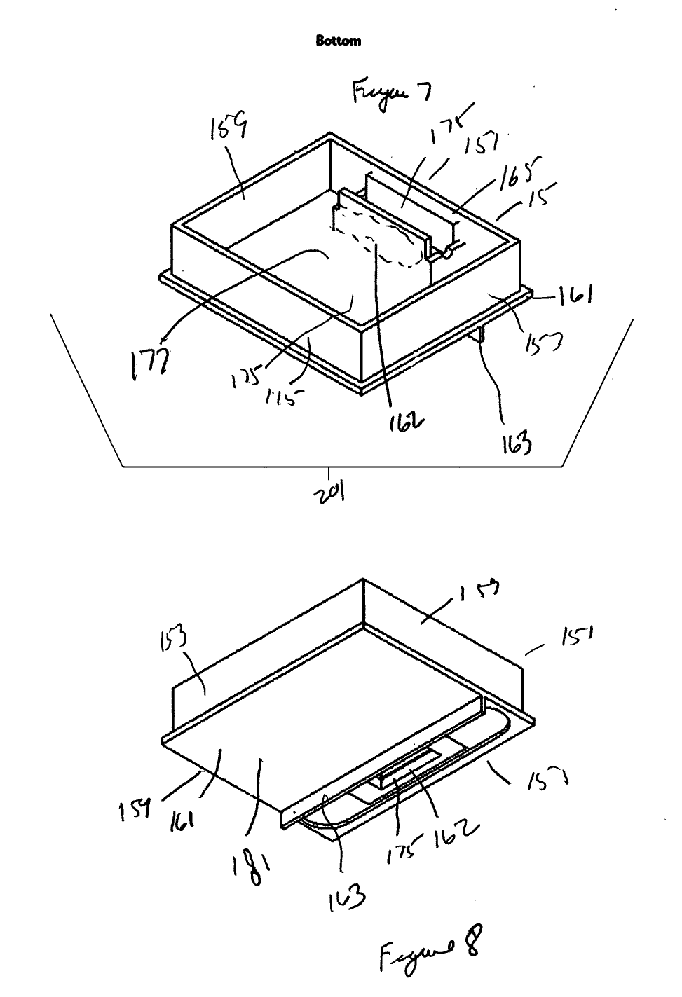

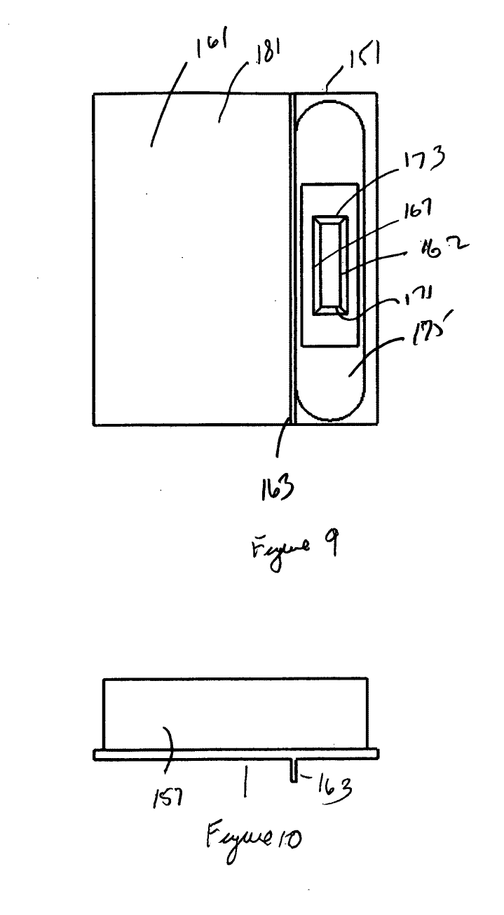

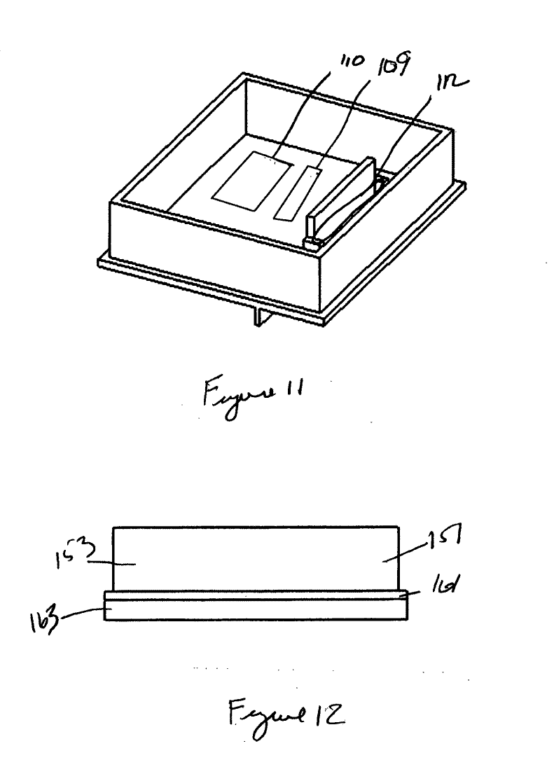

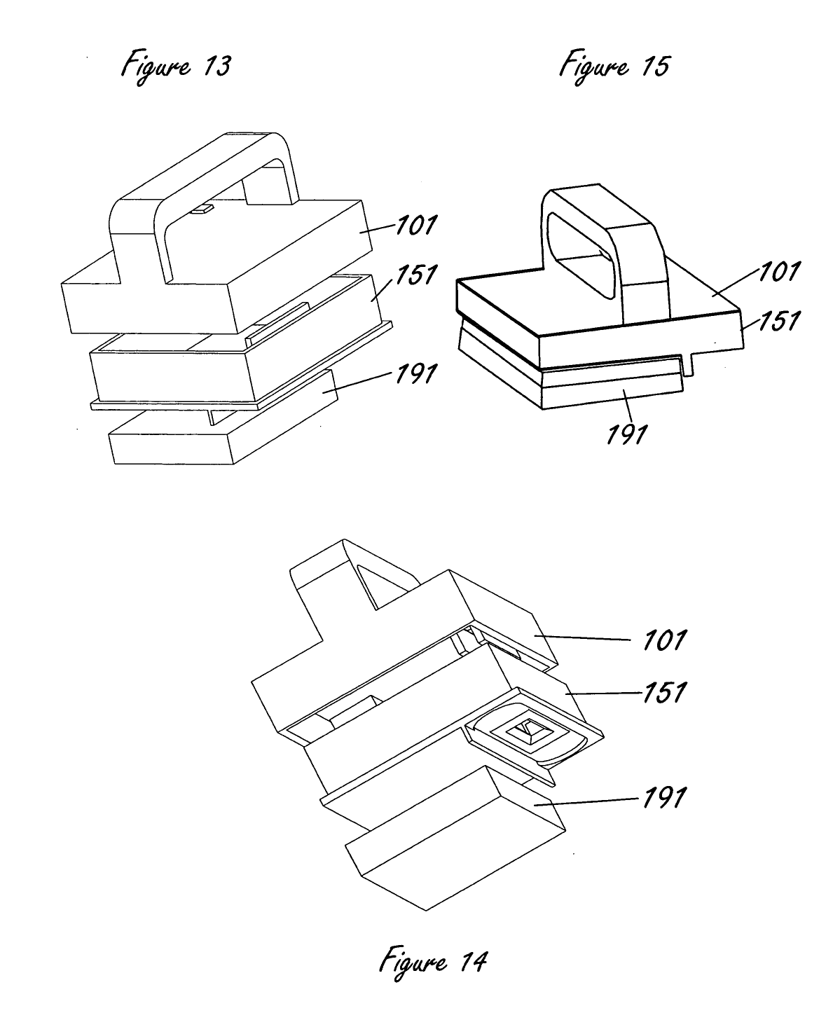

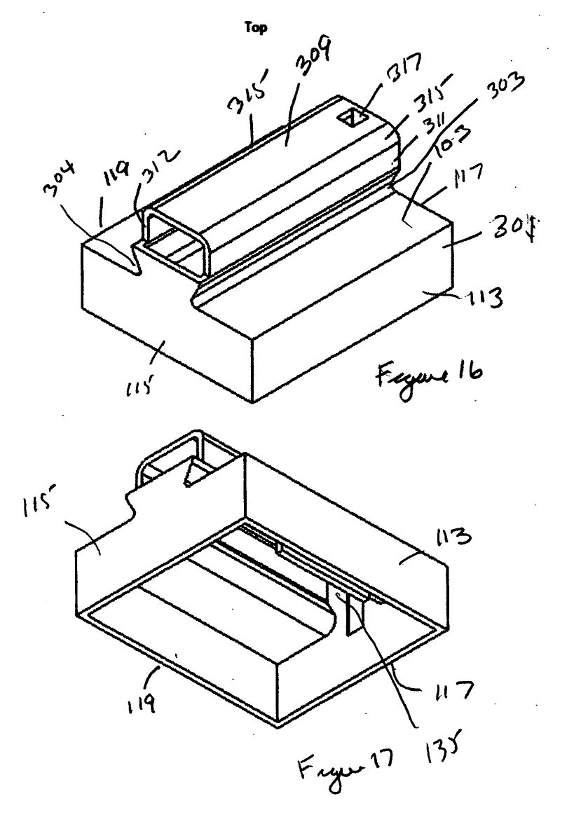

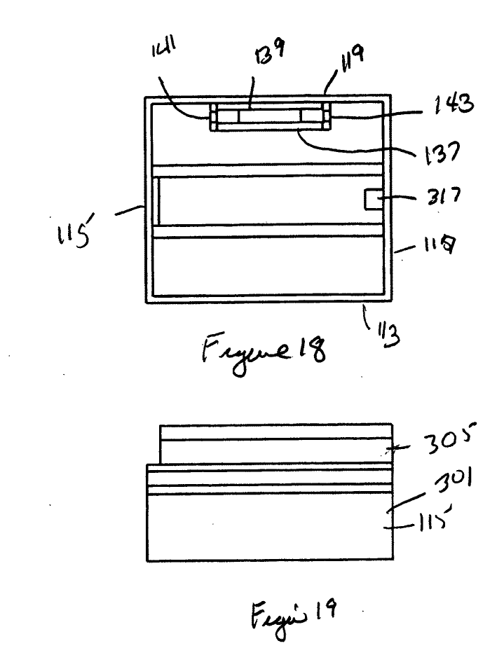

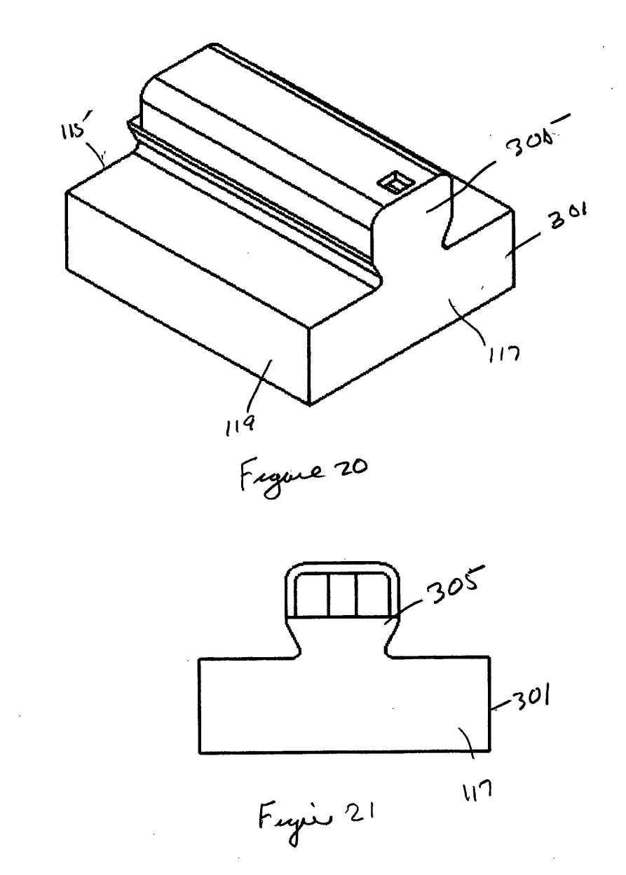

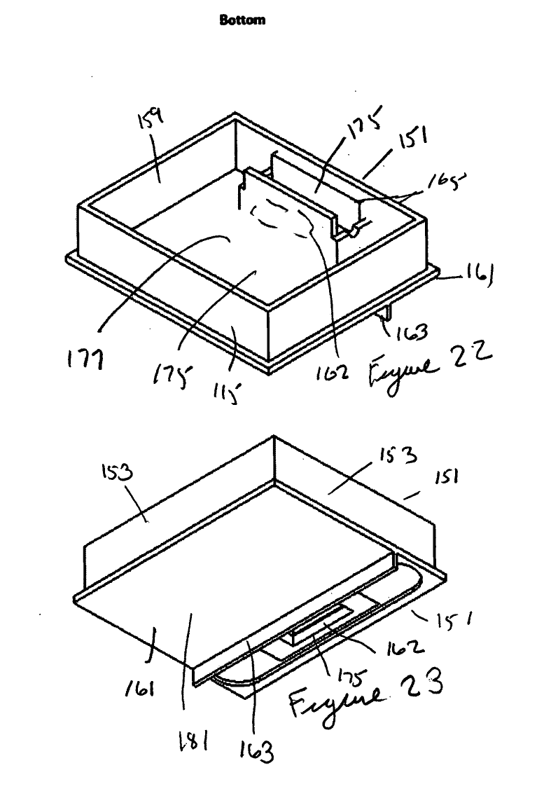

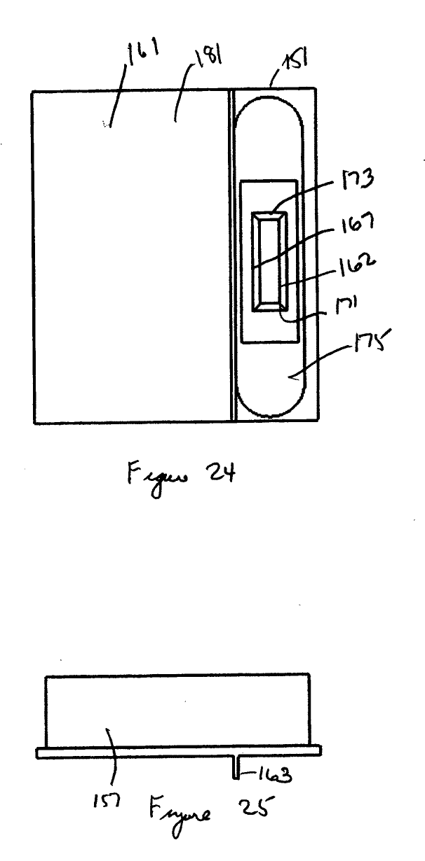

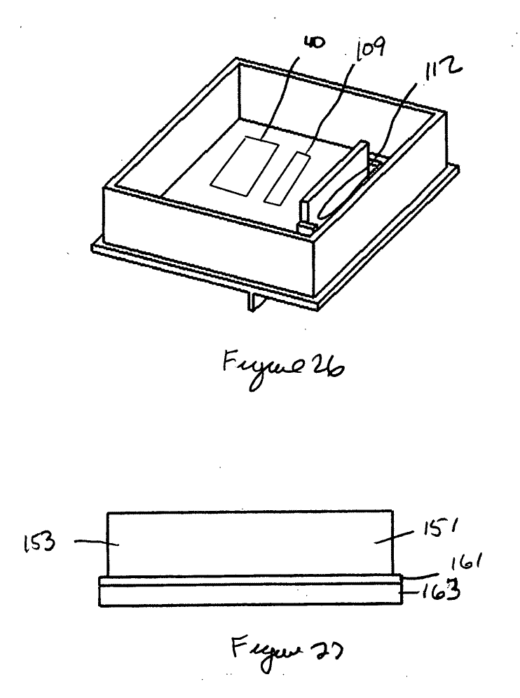

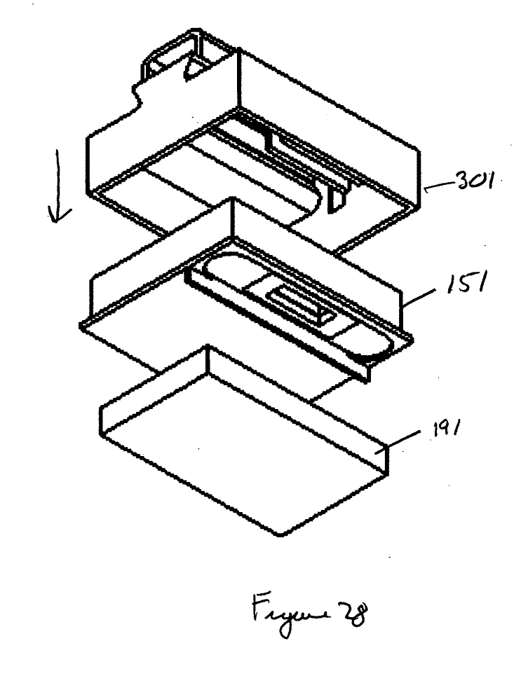

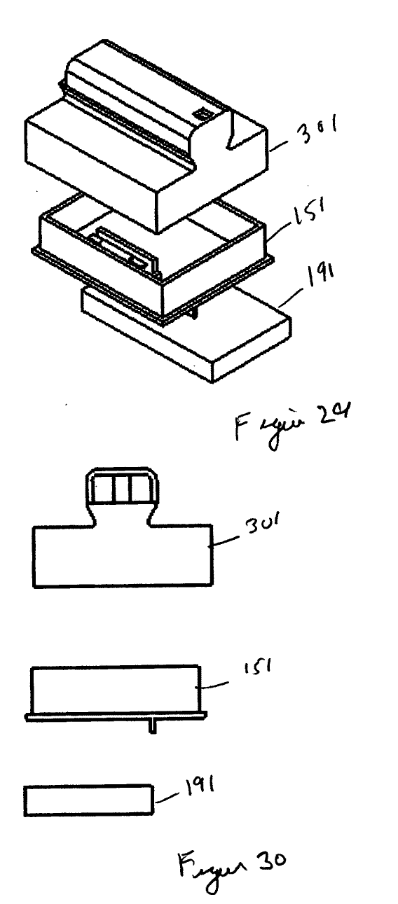

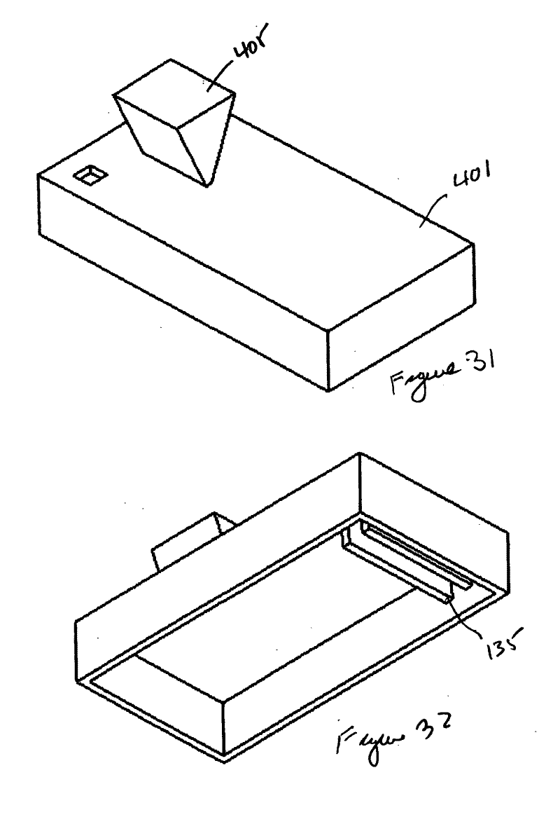

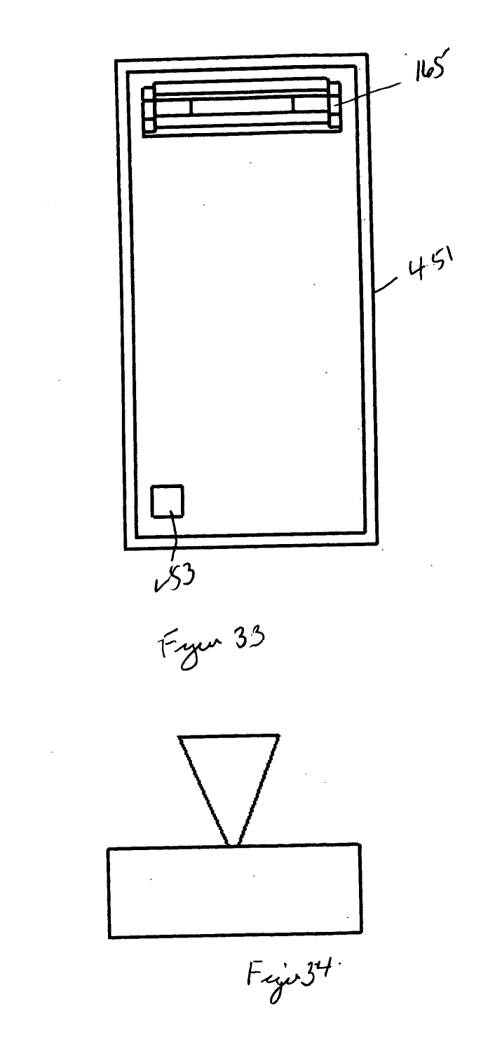

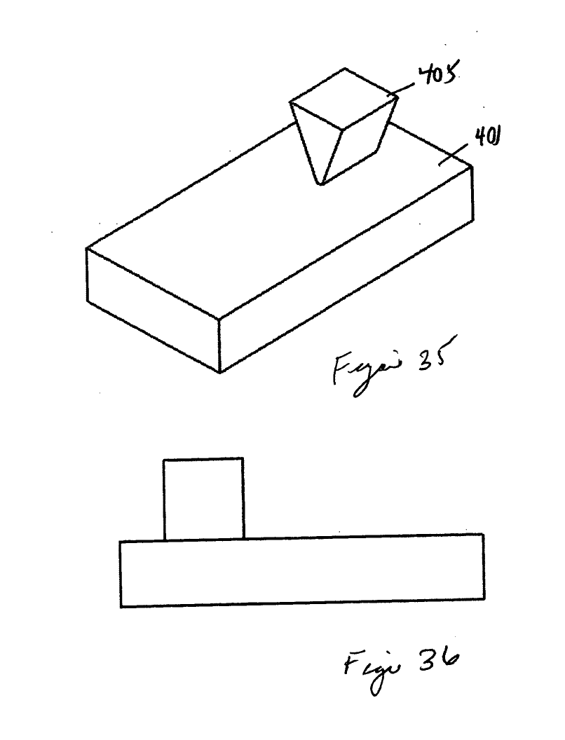

FIG. 3 illustrates a bottom view of the top member 101 and illustrates the front wall 113, the rear wall 119 the first sidewall 115 and the second sidewall 117 of the top member 101. FIG. 3 additionally illustrates the aperture 111, the first enclosure 135 which may include a first sidewall 137, an opposing second sidewall 139, a first end wall 141 and a second end wall 143. The first enclosure 135 may be connected to the bottom surface 131 of the top wall 103.

FIG. 4 illustrates a side view of the top member 101 and illustrates the handle 105 and the first sidewall 115. The handle 105 may include depressions to accommodate the fingers of the user to grip the handle 105 more comfortably.

FIG. 5 illustrates a perspective back view of the top member 101, illustrates the handle member 105 and illustrates the rear wall 119, the first sidewall 115 and the second sidewall 117.

Alternatively, as shown in FIG. 5, a detachable rod 301 could be detachably connected to the top member 101 for example on the handle member 105 in order to allow the user to operate the cleaning and sterilizing device 100 without bending over.

FIG. 6 illustrates a back view of the top member 101 and illustrates the handle 105 and the rear wall 119.

FIG. 7 illustrates a top perspective view of the bottom member 151 which may cooperate with the top member 101 and illustrates a front wall of 153 and a opposed back wall 155 which may be connected to the front wall 153 by a first side wall 157 and a second side wall 159. The front wall 153 and the back wall 155, the first sidewall 157 and a second sidewall 159 may be connected to a bottom wall 161 which may extend outwards from the front wall 153, the back wall 155, the first sidewall 157 and a second sidewall 159. A second enclosure 165 may include a first sidewall 167 and an opposed second sidewall 169 which may be connected to a first end wall 161 and a second end wall 153. The first sidewall 167, the second sidewall 169, the first end wall 161 and the second end wall 153 may extend upwards, and the first sidewall 167 and the second sidewall 169 may extend beyond the first end wall 161 and the second end wall 153. The second enclosure 165 may be connected to the top surface 175 of the bottom wall 161 and the second enclosure 165 may cooperate with the first enclosure 135 to substantially enclose the sterilizing light of the sterilizing light device 109.

The first sidewall 167, the second sidewall 169 the first end wall 171 and the second end wall 173 may define an aperture 175 for the sterilizing light of the sterilizing light device 109 to shine on the work surface 201. The front wall 153, the back wall 155, the first sidewall 157 and a second sidewall 159 defined a aperture 177 to allow the sterilizing light device 109 to be housed within the bottom member 151.

The front wall 113 cooperates with the front wall 153 and the rear wall 119 may cooperate with the back wall 155 and the first sidewall 115 may cooperate with the first sidewall 157 and the second sidewall 117 may cooperate with the second sidewall 159 to define an enclosed space for the sterilizing light device 109.

The bottom wall 161 may include an aperture 162 which may extend through the bottom wall 161 and be defined by the first sidewall 167, the second sidewall 169, the first end wall 171 and the second end wall 173. The aperture 161 allows the sterilizing light from the sterilizing light device 109 to extend to a work surface 201. The bottom surface 177 may include a dividing wall 163 which may extend downward in the longitudinal direction and may extend substantially parallel to an edge of the bottom surface 117 to define a portion 181 of the bottom surface 177 to cooperate with a sponge or other cleaning device which may be connected to the bottom surface 177 by a fastening device which may include Velcro or other appropriate devices.

As the cleaning and sterilizing device 100 passes over the work surface 201, the light emerging from the sterilizing light device 109 contacts the work surface 201 and kills the germs, bacteria and viruses which may be on the work surface 201. Subsequently, as the cleaning and sterilizing device 100 continues to pass over the work surface 201, the cleaning apparatus such as a sponge cleans the dirt and debris from the work surface 201.

FIG. 8 illustrates a bottom perspective view of the bottom member 151 and illustrates the bottom wall 161 which may include the portion 181 which may be defined by the front wall 153, the opposing back wall 155. FIG. 8 additionally illustrates the first sidewall 157 and the second sidewall 159. FIG. 8 additionally illustrates the dividing wall 163 and the aperture 175 to limit the sanitizing light from the sanitizing light device 109.

FIG. 9 illustrates a bottom view of the bottom member 151 and illustrates the bottom wall 161, the aperture 162 of the bottom wall 161, the dividing wall 163 which may extend downward in order to partition the cleaning apparatus from the light from the sterilizing light device 109 which may be connected to a battery 110 in order to power the sterilizing light device and the sterilizing light bulb which may be a cylinder light bulb 112, FIG. 10 illustrates a side view of the bottom member 151 and illustrates the side wall 153 and the dividing wall 163.

FIG. 11 illustrates a top perspective view of the bottom member 151 and illustrates the sterilizing light device 109 which may be an electronic circuit to control and generate the signals so that the sterilizing light bulb 12 generates sterilizing light.

FIG. 12 illustrates a front view of the bottom member 151 and illustrates the front wall 153 and the dividing wall 163, and FIG. 12 additionally illustrates the bottom wall 161.

FIG. 13 illustrates a perspective view of the top member 101, the bottom member 151 and the cleaning apparatus 191.

FIG. 14 illustrates a perspective view of the top member 101, the bottom member 151 and the cleaning apparatus 191.

FIG. 15 illustrates a perspective view of the top member 101, the bottom member 151 and the cleaning apparatus 191.

FIG. 16 illustrates a perspective view of the top member 301 which may include a handle 305 which may extend along the longitudinal direction of the top member 301 and may be substantially centered on the top member 301.

The top member 301 may include a front wall 113 and a opposed rear wall 119 which may be connected to a first sidewall 115 and which may be connected to a second sidewall 117. The front wall 113, the rear wall 119, the first sidewall 115 and the second sidewall 117 may be connected to a top wall 103 which may be connected to a handle 305. The handle 305 may have a top surface 309 which may be connected to a first curved surface 315 and a second curved surface 316 which may be connected to a first side surface 311 and a second side surface 312, respectively. The first side surface 311 may be connected to a first outward inclined surface 303, and the second side surface 312 may be connected to a second outward inclined surface 304. The first outward inclined surface 303 and the second outward inclined surface 304 may be connected to the top wall 103. The first end surface 313 and the second end surface 314 may be connected to the top surface 309, the side surface 311, 312 the outward inclined surface 303, 304, the curved surface 315, 316 and the top surface 309.

The handle 305 may be hollow in order to store the sterile light device 109 which may include the circuit and battery. The top surface 309 may include a aperture 317 to access a switch for the sterile light device 109.

FIG. 17 illustrates that the front wall 113 may be connected to the first sidewall 115 which may be connected to the front wall 113 and the rear wall 119 and which may be opposed to the second side wall 117 which may be connected to the front wall 113 and the rear wall 119. The front wall 113, the rear wall 119, the first sidewall 115 and the second sidewall 119 may define an aperture 113 to cooperate with the bottom member 151.

FIG. 17 illustrates the first enclosure 135 as illustrated in FIG. 2.

FIG. 18 illustrates a bottom view of the top member 301 and illustrates the front wall 113, the rear wall 119 the first sidewall 115 and the second sidewall 117 of the top member 101. FIG. 18 additionally illustrates the aperture 111, the first enclosure 135 which may include a first sidewall 137, an opposing second sidewall 139, a first end wall 141 and a second end wall 143. The first enclosure 135 may be connected to the bottom surface 131 of the top wall 103.

FIG. 19 illustrates a side view of the top member 301 and illustrates the handle 305 and the side wall 119.

FIG. 20 illustrates a perspective back view of the top member 301, illustrates the handle member 305 and illustrates the rear wall 119, the first sidewall 115 and the second sidewall 117.

FIG. 21 illustrates a side view of the top member 301 and illustrates the handle member 305, the top member 301 and the side wall 117.

FIG. 22 illustrates a top perspective view of the bottom member 151 which may cooperate with the top member 101 and illustrates a front wall of 153 and a opposed back wall 155 which may be connected to the front wall 153 by a first side wall 157 and a second side wall 159. The front wall 153 and the back wall 155, the first sidewall 157 and a second sidewall 159 may be connected to a bottom wall 161 which may extend outwards from the front wall 153, the back wall 155, the first sidewall 157 and a second sidewall 159. A second enclosure 165 may include a first sidewall 167 and an opposed second sidewall 169 which may be connected to a first end wall 161 and a second end wall 153. The first sidewall 167, the second sidewall 169, the first end wall 161 and the second end wall 153 may extend upwards, and the first sidewall 167 and the second sidewall 169 may extend beyond the first end wall 161 and the second end wall 153. The second enclosure 165 may be connected to the top surface 175 of the bottom wall 161 and the second enclosure 165 may cooperate with the first enclosure 135 to substantially enclose the sterilizing light of the sterilizing light device 109.

The first sidewall 167, the second sidewall 169 the first end wall 171 and the second end wall 173 may define an aperture 175 for the sterilizing light of the sterilizing light device 109 to shine on the work surface 201. The front wall 153, the back wall 155, the first sidewall 157 and a second sidewall 159 defined a aperture 177 to allow the sterilizing light device 109 to be housed within the bottom member 151.

The front wall 113 cooperates with the front wall 153 and the rear wall 119 may cooperate with the back wall 155 and the first sidewall 115 may cooperate with the first sidewall 157 and the second sidewall 117 may cooperate with the second sidewall 159 to define an enclosed space for the sterilizing light device 109.

The bottom wall 161 may include an aperture 162 which may extend through the bottom wall 161 and be defined by the first sidewall 167, the second sidewall 169, the first end wall 171 and the second end wall 173. The aperture 161 allows the sterilizing light from the sterilizing light device 109 to extend to a work surface 201. The bottom surface 177 may include a dividing wall 163 which may extend downward in the longitudinal direction and may extend substantially parallel to an edge of the bottom surface 117 to define a portion 181 of the bottom surface 177 to cooperate with a sponge or other cleaning device which may be connected to the bottom surface 177 by a fastening device which may include Velcro or other appropriate devices.

FIG. 23 illustrates a bottom perspective view of the bottom member 151 and illustrates the bottom wall 161 which may include the portion 181 which may be defined by the front wall 153, the opposing back wall 155. FIG. 23 additionally illustrates the first sidewall 157 and the second sidewall 159. FIG. 8 additionally illustrates the dividing wall 163 and the aperture 175 to limit the sanitizing light from the sanitizing light device 109.

FIG. 24 illustrates a bottom view of the bottom member 151 and illustrates the bottom wall 161, the aperture 162 of the bottom wall 161, the dividing wall 163 which may extend downward in order to partition the cleaning apparatus from the light from the sterilizing light device 109 which may be connected to a battery 110 in order to power the sterilizing light device and the sterilizing light bulb which may be a cylinder light bulb 112. The bottom member 161 may include additional reflective surfaces in order to direct the light more intensely to the work surface 201.

FIG. 25 illustrates a side view of the bottom member 151 and illustrates the side wall 153 and the dividing wall 163.

FIG. 26 illustrates a top perspective view of the bottom member 151 and illustrates the sterilizing light device 109 which may be an electronic circuit to control and generate the signals so that the sterilizing light bulb 12 generates sterilizing light.

FIG. 27 illustrates a front view of the bottom member 151 and illustrates the front wall 153 and the dividing wall 163, and FIG. 12 additionally illustrates the bottom wall 161.

FIG. 28 illustrates a perspective view of the top member 301, the bottom member 151 and the cleaning apparatus 191.

FIG. 29 illustrates a perspective view of the top member 301, the bottom member 151 and the cleaning apparatus 191.

FIG. 30 illustrates a side view of the top member 131, the bottom member 151 and the cleaning apparatus 191.

FIG. 31 illustrates another embodiment of the present invention which illustrates a substantially triangular handle 405 which is mounted on the top member 401.

FIG. 32 illustrates that the first enclosure 135 is positioned at an end of the top member 401.

FIG. 33 illustrates the bottom member 451 which may cooperate with the top member 401 and illustrates the second enclosure 165 to cooperate with the first enclosure 135. FIG. 33 illustrates an aperture 453 to operate the switch for the sterilizing light device 109.

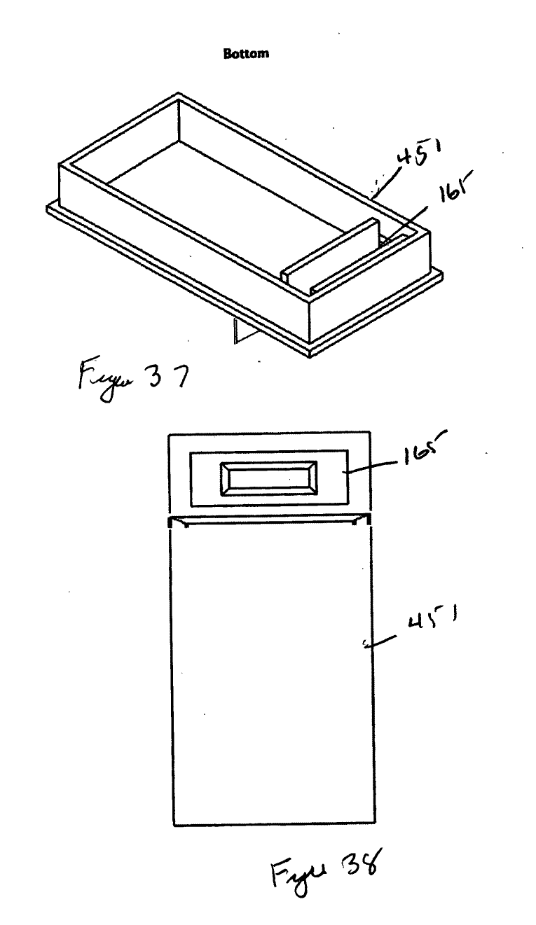

FIG. 34 illustrates a side view of the top member 401 and handle 405.

FIG. 35 illustrates a perspective view of the top member 401 and handle 405.

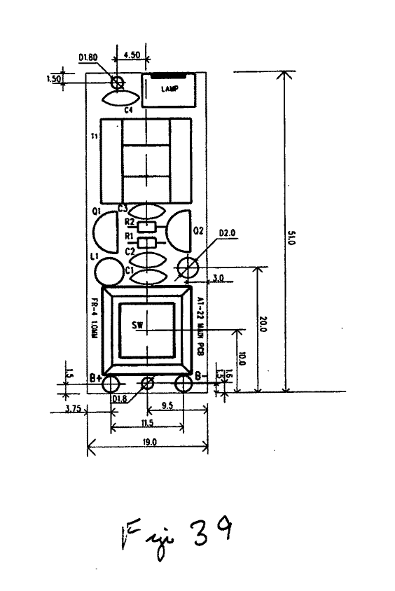

FIG. 36 illustrates a side view of the top member 401 and handle 405.

FIG. 37 illustrates a perspective view of the bottom member 451 and a second enclosure 165.

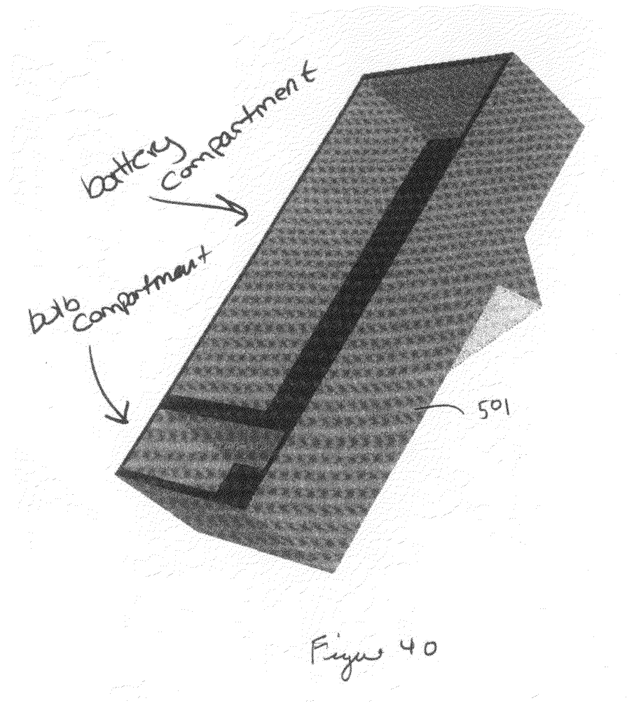

FIG. 38 illustrates a bottom view of the bottom member 451 and the second enclosure 165.

FIG. 39 illustrates a circuit diagram of the sterilizing light device 109.

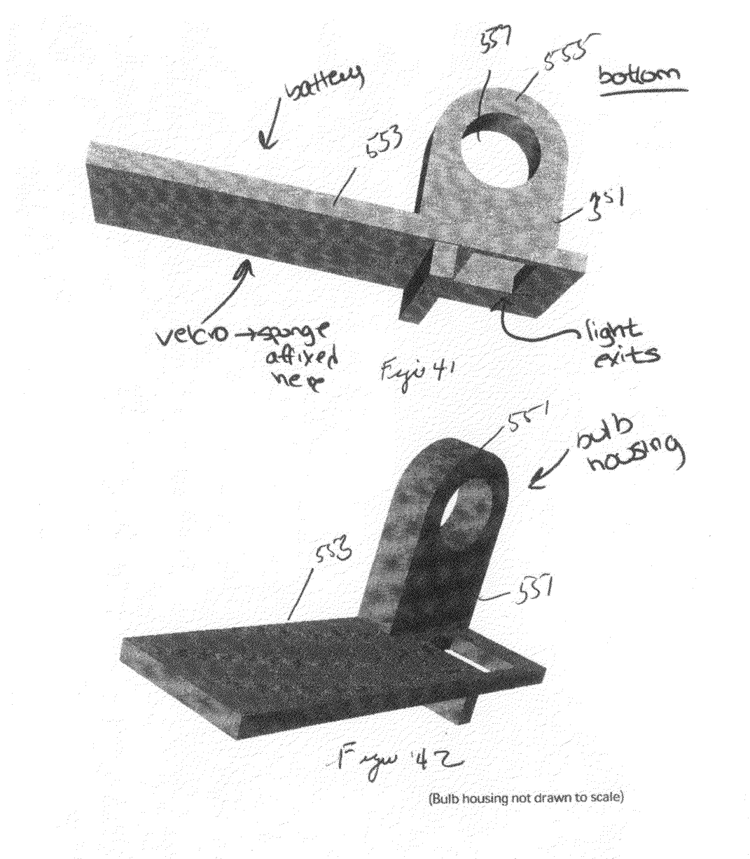

FIG. 40 illustrates a alternative top member 501 and illustrates a battery compartment and bow compartment within the top member 501.

FIG. 41 illustrates a alternative bottom member 551 which includes a platform 553 for the battery and sterilizing light device 109 and includes an upward extending post 555 having a aperture 557 to mount a light bulb of the sterilizing light device 109.

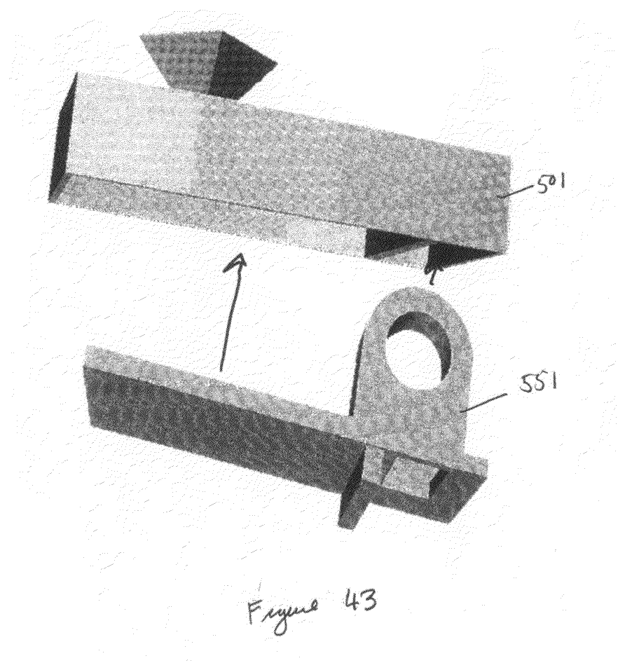

FIG. 42 illustrates a perspective view of the bottom member 551.

FIG. 43 illustrates the top member 501 cooperating with the bottom member 551.

Like numbers have been assigned to similar elements having similar functions.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A cleaning and sterilizing device to clean and sterilize a work surface, comprising:
a top member;
a bottom member to cooperate with the top member to provide a container for a sterilizing light device to provide sterilizing light for the work surface;
wherein the top member includes a first enclosure and the bottom member includes a second enclosure to cooperate with the first enclosure to substantially cover a light bulb to generate a sterilizing light for the work surface and wherein a cleaning apparatus is detachably connected to the bottom member to simultaneously clean the work surface as the sterilizing light is sterilizing the work surface.

2) A cleaning and sterilizing device to clean and sterilize a work surface as in claim 1, wherein the sterilizing light is an ultraviolet light.

3) A cleaning and sterilizing device to clean and sterilize a work surface as in claim 1, wherein the cleaning apparatus includes a sponge detachably connected to the bottom member.

4) A cleaning and sterilizing device to clean and sterilize a work surface as in claim 3, wherein the sponge is connected to the bottom member with a Velcro hooks and loops.

5) A cleaning and sterilizing device to clean and sterilize a work surface as in claim 3, wherein the sponge is connected to the bottom surface of the bottom member.

6. A cleaning and sterilizing device to clean and sterilize a work surface as in claim 3, wherein the bottom member includes a dividing wall to separate the sponge from the sterilizing light.
